(19) United States
(12) Patent Application Publication
(10) Pub. No.: US 2026/0230993 A1
(43) Pub. Date: Aug. 6, 2026

(54) SELECTION OF A RELAY IN SIDELINK COMMUNICATION

(71) Applicant: TOYOTA JIDOSHA KABUSHIKI KAISHA, Toyota-shi, Aichi-ken (JP)

(72) Inventor: Wen-Yao CHANG, New Taipei City (TW)

(73) Assignee: TOYOTA JIDOSHA KABUSHIKI KAISHA, Toyota-shi, Aichi-ken (JP)

(21) Appl. No.: 19/151,918

(22) PCT Filed: Jan. 23, 2024
(86) PCT No.: PCT/JP2024/001875

§ 371 Date: Jul. 29, 2025

Related U.S. Application Data
(60) Provisional application No. 63/482,128, filed on Jan. 30, 2023.

(51) Int. Cl. H04W 40/22; H04W 40/12; H04W 40/24

(52) U.S. Cl. CPC H04W 40/22; H04W 40/12; H04W 40/246

(57) ABSTRACT

Disclosed are methods, apparatuses, and systems for relay selection in a communication. The method includes: receiving, by a base station, one or more discovery messages communicated between the UE and one or more candidate relays, the UE and the one or more candidate relays being attached to the base station; receiving, by the base station, one or more measurements on one or more signals transmitted between the base station and the one or more candidate relays; determining, by the base station, whether the one or more candidate relays satisfy relay selection criteria based on at least one of: the one or more discovery messages, or the one or more measurements; and selecting, by the base station and based on the determining, at least one relay from the one or more candidate relays for a multi-path transmission for the UE.

### CROSS-REFERENCE TO RELATED PATENT APPLICATION

This application claims the benefit of U.S. Provisional Application No. 63/482,128, filed on Jan. 30, 2023, entitled “SELECTION OF RELAY UES FOR MULTI-PATH SIDELINK RELAYING COMMUNICATION,” the entirety of which is incorporated by reference herein.

### TECHNICAL FIELD

Apparatuses and methods consistent with the present disclosure relate generally to communications, more specifically, methods, systems, and devices for multi-path transmission in communications.

### BACKGROUND ART

A user equipment (UE) may adopt a muti-path transmission to communicate with a base station to improve reliability and efficiency of the communication. The multi-path transmission may include a direct path between the UE and the base station, and one or more indirect paths between the UE and the base station via one or more selected relays. The relays can be one or more other UEs in the communication. For an indirect path, the communication between the UE and the base station includes a first link between the UE and the relay, and a second link between the relay and the base station. Selecting a relay that can ensure the communication quality of the both links is important for the overall communication quality of the indirect path. However, relay selection (or reselection) is challenging because the communication characteristics of the first link between the UE and the relay are unknown to the base station, and the communication characteristics of the second link between the relay and the base station are unknown to the UE. That is, neither the UE nor the base station has the overall knowledge of the characteristics of both the first and second links, causing inaccuracy in relay selection, thereby degrading the quality of the indirect path. Systems and methods for reliable and accurate relay selection (or reselection) for a multi-path transmission are desired.

### SUMMARY OF INVENTION

According to some embodiments of the present disclosure, there is provided a base station in a communication system. The base station includes a memory storing an instruction; and a processor configured to execute the instruction stored in the memory to: receive one or more discovery messages communicated between the UE and the one or more candidate relays, the UE and the one or more candidate relays being attached to the base station; receive one or more measurements on one or more signals transmitted between the base station and the one or more candidate relays; determine whether the one or more candidate relays satisfy relay selection criteria based on at least one of: the one or more discovery messages, or the one or more measurements; and select at least one relay from the one or more candidate relays for a multi-path transmission for the UE.

According to some embodiments of the present disclosure, there is provided a UE for a communication. The UE includes a memory storing an instruction; and a processor configured to execute the instruction stored in the memory to: receive, from one or more candidate relays attached to the base station, one or more discovery messages, the one or more discovery messages comprising at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission; transmit, to the base station, the one or more discovery messages; receive, from the base station, at least one relay selected from the one or more candidate relays; and trigger a multi-path transmission, the multi-path transmission comprising a direct path between the UE and the base station, and an indirect path between the UE and the base station via the selected at least one relay.

According to some embodiments of the present disclosure, there is provided a UE for a communication. The UE includes a memory storing an instruction; and a processor configured to execute the instruction stored in the memory to: transmit, to the base station, a relay selection request such that the relay selection request is forwarded to one or more candidate relays attached to the base station; transmit, to the one or more candidate relays, one or more relay selection response messages, the one or more relay selection response messages comprising one or more link quality parameters for one or more links between the UE and the one or more candidate relays; receive, from the base station, at least one relay selected from the one or more candidate relays; and trigger a multi-path transmission, the multi-path transmission comprising a direct path between the UE and the base station, and an indirect path between the UE and the base station via the selected at least one relay.

According to some embodiments of the present disclosure, there is provided a relay for a UE in a communication. The relay includes a memory storing an instruction; and a processor configured to execute the instruction stored in the memory to: transmit, to the UE, one or more discovery messages, the one or more discovery messages comprising at least one of: (1) one or more link quality parameters for one or more links between the UE and the relay, (2) an attachment status parameter indicating attachment of the relay to the base station, or (3) an availability status parameter indicating availability of the relay for multi-path transmission; and transmit, to the base station, one or more measurements on one or more signals transmitted between the base station and the relay, the one or more measurements comprising at least one of: (1) one or more link quality parameters for one or more links between the base station and the relay, or (2) an availability status parameter indicating availability of the relay for the multi-path transmission.

According to some embodiments of the present disclosure, there is provided a relay for a UE in a communication. The relay includes a memory storing an instruction; and a processor configured to execute the instruction stored in the memory to: receive, from the base station, a relay selection request; receive, from the UE, one or more relay selection response messages, the one or more relay selection response messages including one or more link quality parameters for one or more links between the UE and the relay; transmit, to the base station, the received one or more relay selection response messages; and transmit, to the base station, one or more measurements on one or more signals transmitted between the base station and the relay, the one or more measurements comprising at least one of: (1) one or more link quality parameters for one or more links between the base station and the relay, or (2) one or more availability status parameters indicating availability of the relay for a multi-path transmission.

According to some embodiments of the present disclosure, there is provided a method for selecting at least one relay for a UE in a communication system. The method includes receiving, by a base station, one or more discovery messages communicated between the UE and one or more candidate relays, the UE and the one or more candidate relays being attached to the base station; receiving, by the base station, one or more measurements on one or more signals transmitted between the base station and the one or more candidate relays; determining, by the base station, whether the one or more candidate relays satisfy relay selection criteria based on at least one of: the one or more discovery messages, or the one or more measurements; and selecting, by the base station and based on the determining, at least one relay from the one or more candidate relays for a multi-path transmission for the UE.

According to some embodiments of the present disclosure, there is provided a method for a UE, the UE being attached to a base station in a communication system. The method includes receiving, from one or more candidate relays attached to the base station, one or more discovery messages, the one or more discovery messages comprising at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission; transmitting, to the base station, the one or more discovery messages; receiving, from the base station, information regarding at least one relay selected from the one or more candidate relays; and triggering a multi-path transmission, the multi-path transmission comprising a direct path between the UE and the base station, and an indirect path between the UE and the base station via the selected at least one relay.

According to some embodiments of the present disclosure, there is provided a method for a UE, the UE being attached to a base station in a communication system. The method includes transmitting, to the base station, a relay selection request such that the relay selection request is forwarded to one or more candidate relays attached to the base station; transmitting, to the one or more candidate relays, one or more relay selection response messages, the one or more relay selection response messages comprising one or more link quality parameters for one or more links between the UE and the one or more candidate relays; receiving, from the base station, information regarding at least one relay selected from the one or more candidate relays; and triggering a multi-path transmission, the multi-path transmission comprising a direct path between the UE and the base station, and an indirect path between the UE and the base station via the selected at least one relay.

According to some embodiments of the present disclosure, there is provided a method for a relay for a UE in a communication system, the relay and the UE being attached to a base station. The method includes transmitting, to the UE, one or more discovery messages, the one or more discovery messages comprising at least one of: (1) one or more link quality parameters for one or more links between the UE and the relay, (2) an attachment status parameter indicating attachment of the relay to the base station, or (3) an availability status parameter indicating availability of the relay for multi-path transmission; and transmitting, to the base station, one or more measurements on one or more signals transmitted between the base station and the relay, the one or more measurements comprising at least one of: (1) one or more link quality parameters for one or more links between the base station and the relay, or (2) an availability status parameter indicating availability of the relay for the multi-path transmission.

According to some embodiments of the present disclosure, there is provided a method for a relay for a UE in a communication system, the relay and the UE being attached to a base station. The method includes receiving, from the base station, a relay selection request; receiving, from the UE, one or more relay selection response messages, the one or more relay selection response messages including one or more link quality parameters for one or more links between the UE and the relay; transmitting, to the base station, the received one or more relay selection response messages; and transmitting, to the base station, one or more measurements on one or more signals transmitted between the base station and the relay, the one or more measurements comprising at least one of: (1) one or more link quality parameters for one or more links between the base station and the relay, or (2) one or more availability status parameters indicating availability of the relay for a multi-path transmission.

According to some embodiments of the present disclosure, there is provided a non-transitory computer-readable medium storing instructions that are executable by one or more processors of a base station in a communication to perform a method. The method includes receiving one or more discovery messages communicated between the UE and one or more candidate relays, the UE and the one or more candidate relays being attached to the base station; receiving one or more measurements on one or more signals transmitted between the base station and the one or more candidate relays; determining whether the one or more candidate relays satisfy relay selection criteria based on at least one of: the one or more discovery messages, or the one or more measurements; and selecting, based on the determining, at least one relay from the one or more candidate relays for a multi-path transmission for the UE.

According to some embodiments of the present disclosure, there is provided a non-transitory computer-readable medium storing instructions that are executable by one or more processors of a UE in a communication to perform a method. The method includes receiving, from one or more candidate relays attached to the base station, one or more discovery messages, the one or more discovery messages comprising at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission; transmitting, to the base station, the one or more discovery messages; receiving, from the base station, information regarding at least one relay selected from the one or more candidate relays; and triggering a multi-path transmission, the multi-path transmission comprising a direct path between the UE and the base station, and an indirect path between the UE and the base station via the selected at least one relay.

According to some embodiments of the present disclosure, there is provided a non-transitory computer-readable medium storing instructions that are executable by one or more processors of a UE in a communication to perform a method. The method includes transmitting, to the base station, a relay selection request such that the relay selection request is forwarded to one or more candidate relays attached to the base station; transmitting, to the one or more candidate relays, one or more relay selection response messages, the one or more relay selection response messages comprising one or more link quality parameters for one or more links between the UE and the one or more candidate relays; receiving, from the base station, information regarding at least one relay selected from the one or more candidate relays; and triggering a multi-path transmission, the multi-path transmission comprising a direct path between the UE and the base station, and an indirect path between the UE and the base station via the selected at least one relay.

According to some embodiments of the present disclosure, there is provided a non-transitory computer-readable medium storing instructions that are executable by one or more processors of a relay for a UE in a communication to perform a method. The method includes transmitting, to the UE, one or more discovery messages, the one or more discovery messages comprising at least one of: (1) one or more link quality parameters for one or more links between the UE and the relay, (2) an attachment status parameter indicating attachment of the relay to the base station, or (3) an availability status parameter indicating availability of the relay for multi-path transmission; and transmitting, to the base station, one or more measurements on one or more signals transmitted between the base station and the relay, the one or more measurements comprising at least one of: (1) one or more link quality parameters for one or more links between the base station and the relay, or (2) an availability status parameter indicating availability of the relay for the multi-path transmission.

According to some embodiments of the present disclosure, there is provided a non-transitory computer-readable medium storing instructions that are executable by one or more processors of a relay for a UE in a communication to perform a method. The method includes receiving, from the base station, a relay selection request; receiving, from the UE, one or more relay selection response messages, the one or more relay selection response messages including one or more link quality parameters for a link between the UE and the relay; transmitting, to the base station, the received one or more relay selection response messages; and transmitting, to the base station, one or more measurements on one or more signals transmitted between the base station and the relay, the one or more measurements comprising at least one of: (1) one or more link quality parameters for one or more links between the base station and the relay, or (2) one or more availability status parameters indicating availability of the relay for a multi-path transmission.

### BRIEF DESCRIPTION OF DRAWINGS

FIG. 1 is a schematic diagram illustrating a multi-path transmission in a communication system, consistent with some embodiments of the present disclosure.

FIG. 2 is a schematic diagram illustrating challenges in relay selection (or reselection) for a multi-path transmission.

FIG. 3 is a schematic diagram illustrating a method for relay selection (or reselection), consistent with some embodiments of the present disclosure.

FIG. 4 is a schematic diagram illustrating a first approach for relay selection (or reselection), consistent with some embodiments of the present disclosure.

FIG. 5 is a schematic diagram illustrating a second approach for relay selection (or reselection), consistent with some embodiments of the present disclosure.

FIG. 6 is a schematic diagram illustrating a method for a base station for a multi-path communication, consistent with some embodiments of the present disclosure.

FIG. 7 is a schematic diagram illustrating a method for a UE for a multi-path communication, consistent with some embodiments of the present disclosure.

FIG. 8 is a schematic diagram illustrating a method for a UE for a multi-path communication, consistent with some embodiments of the present disclosure.

FIG. 9 is a schematic diagram illustrating a method for a relay for a UE in a communication, consistent with some embodiments of the present disclosure.

FIG. 10 is a schematic diagram illustrating a method for a relay for a UE in a communication, consistent with some embodiments of the present disclosure.

FIG. 11 is a block diagram of a UE, consistent with some embodiments of the present disclosure.

### DESCRIPTION OF EMBODIMENTS

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the present disclosure. Instead, they are merely examples of systems, apparatuses, and methods consistent with aspects related to the present disclosure as recited in the appended claims.

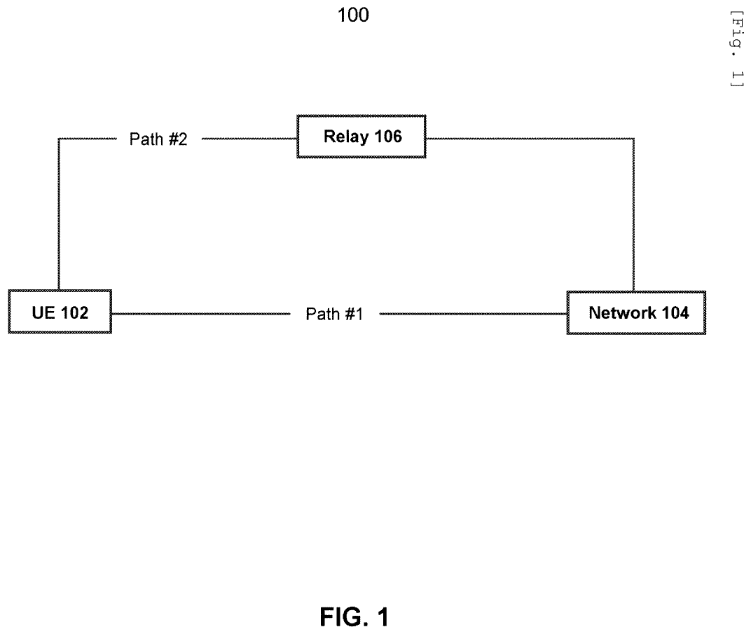

FIG. 1 is a schematic diagram illustrating a multi-path transmission in a communication system, consistent with some embodiments of the present disclosure. Referring to

FIG. 1, a communication system 100 includes a UE 102, a network 104, and a relay 106. The network may be a base station (e.g., gNB). The relay 106 may be a UE-to-network (U2N) relay. The relay 106 can be another UE in the communication system that is attached to the network 104. As shown in FIG. 1, the UE 102 may be connected to the network 104 using a direct path (path #1) and an indirect path (path #2) via the relay 106 for a multi-path transmission. In some embodiments, there can be multiple indirect paths via multiple relays.

Referring to FIG. 1, when the relay 106 is attached to the network 104, for example, using Uu interface, the information on the link between the relay 106 and the network 104 is known to the network 104. The information on the link between the relay 106 and the network 104 may include, for example, Uu quality of service (QoS) characteristics (e.g., channel condition) and the availability status (e.g., network attachment, workload) of the relay. The availability status may include, for example, information on whether the relay 106 is attached to the network 104, and/or whether the workload of the relay 106 allows the relay 106 to perform the function of a relay for the UE 102. The information on the link between the relay 106 and the network 104 would be beneficial in determining whether to select (or reselect) the relay 106 as a relay for a multi-path transmission for the UE 102. However, under the existing technology, the information on the link between the relay 106 and the network 104 is unknown to the UE 102.

Also, when the UE 102 discovers and selects the relay 106 on its own, the UE 102 and the relay 106 form a link, for example, via PC5 interface. The information on the link between the UE 102 and the relay 106 is known to the UE 102. The information on the link between the UE 102 and the relay 106 may include, for example, PC5 QoS characteristics, the availability status of the relay 106, etc. The availability status of the relay 106 may include, for example, information on network attachment (e.g., whether or not the relay 106 is attached to the network 104), and/or information on the workload of the relay 106. However, under the existing technology, the information on the link between the UE 102 and the relay 106 is unknown to the network 104. Therefore, the end-to-end quality of the indirect path (path #2) may not be guaranteed, thereby degrading the overall performance of the multi-path transmission.

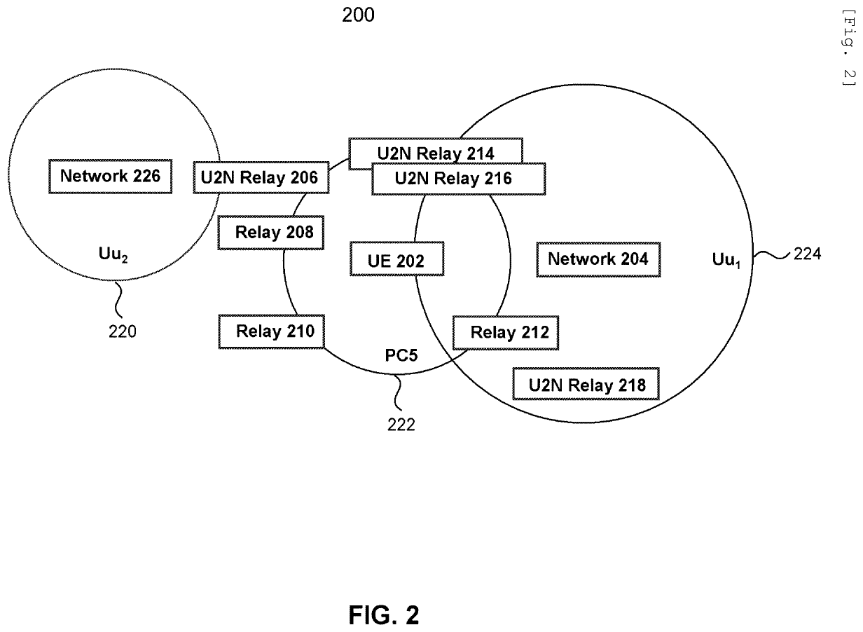

FIG. 2 is a schematic diagram illustrating challenges in relay selection (or reselection) for a multi-path transmission. Referring to FIG. 2, a communication system includes a UE 202, two networks 204 and 226, four U2N relays 206, 214, 216, and 218, and three non-U2N relays 208, 210, and 212. The two networks 204 and 226 can be base stations (e.g., gNBs). The non-U2N relays 208, 210, and 212 are not attached to any networks. The communication system 200 includes two types of connections (links): a UE-to-UE connection via PC5 interface indicated by a circle 222, and a UE-to-network connection via Uu interface indicated by a circle 224 (Uu1 interface) and a circle 220 (Uu2 interface). Among the four U2N relays, the U2N relays 214, 216, and 218 are attached to the network 204 via Uu1, and the U2N relay 206 is attached to the network 226 via Uu2. The UE 202 is attached to the network 204 via the Uu1 interface. Through the PC5 connection, the UE 202 may find six candidate relays on its own, they are: the U2N relay 206, the relay 208, the relay 210, the relay 212, the U2N relay 216, and the U2N relay 214. Among these six candidate relays, only three relays (214, 216, and 206) are attached to a network (204 or 226), and thus, can be any U2N relays; and the remaining three relays (208, 210, and 212) cannot be a U2N relay. And among the three U2N relays, the U2N relay 206 is attached to a network 226, which is not the same network to which the UE 202 is attached (network 204). Thus, the U2N relay 206 cannot be a candidate U2N relay for the UE 202. That is, among the six relays that the UE 202 finds, only two of them can be candidate U2N relays for the UE 202, indicating challenges in selecting relays. At least some embodiments of the present disclosure address the above noted challenges by providing a relay selection (or reselection) method with the assistance (or guidance) of the network, to facilitate end-to-end quality of the indirect path in multi-path transmission using U2N relay.

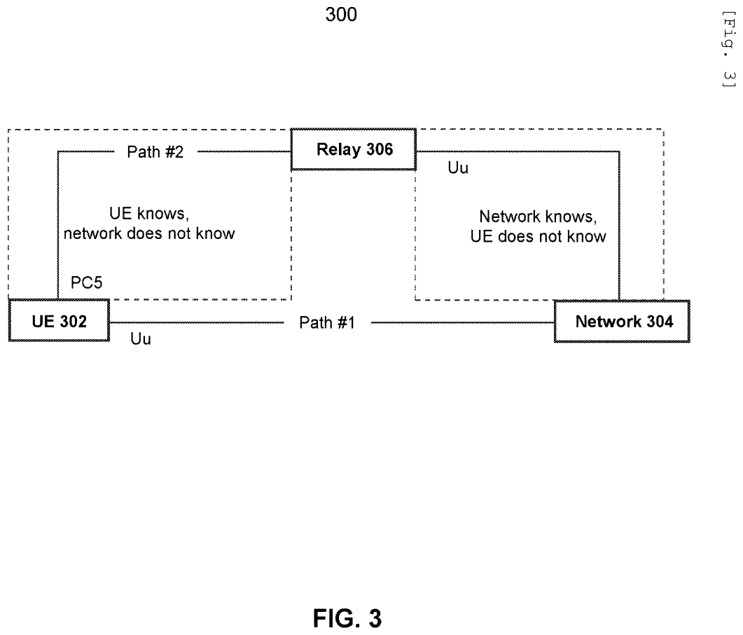

FIG. 3 is a schematic diagram illustrating a method for relay selection (or reselection), consistent with some embodiments of the present disclosure. Referring to FIG. 3, a communication system 300 includes a UE 302, a network 304, and a relay 306. The UE 302 can be any device, for example, a mobile phone, a wireless handheld device, wireless personal device, a computer, a laptop computer, a vehicle, a component mounted in a vehicle, or any other form. The network 304 can be any network, for example, a base station. The base station can be any base station currently existing (e.g., eNB, gNB) for long term evolution (LTE) or new radio (NR), or a base station for a future generation (6^{th} generation (6G), or any other future generation) radio access technology (RAT). The relay 306 may be a U2N relay. In some embodiments, the U2N relay can be another UE. In some embodiments, the UE 302 is a vehicle in a vehicle-to-everything (V2X) communication, and the U2N relay is a road side unit. As shown in FIG. 3, the UE 302 may be connected to the network 304 using a direct path (path #1) and an indirect path (path #2) via the relay 306 for a multi-path transmission. In some embodiments, the UE 302 uses multiple indirect paths to communicate with the base station via multiple relays.

Referring to FIG. 3, the indirect path #2 includes two links: a first link between the UE 302 and the relay 306, for example, via PC5 interface, and a second link between the relay 306 and the network 304, for example, via Uu interface. The characteristics (e.g., link QoS parameters, attachment, availability, etc.) of the first link between the UE 302 and the relay 306 are known to UE 302, but are not known to the network 304. On the other hand, the characteristics of the link between the relay 306 and the network 304 are known to the network 304, but are not known to the UE 302. At least some embodiments of the present disclosure are directed to combining a priori knowledge of UE 302 and the network 304, and assisting selection (or reselection) of suitable U2N relays in consideration of end-to-end quality of the indirect path #2, thereby improving reliability and accuracy of the communication.

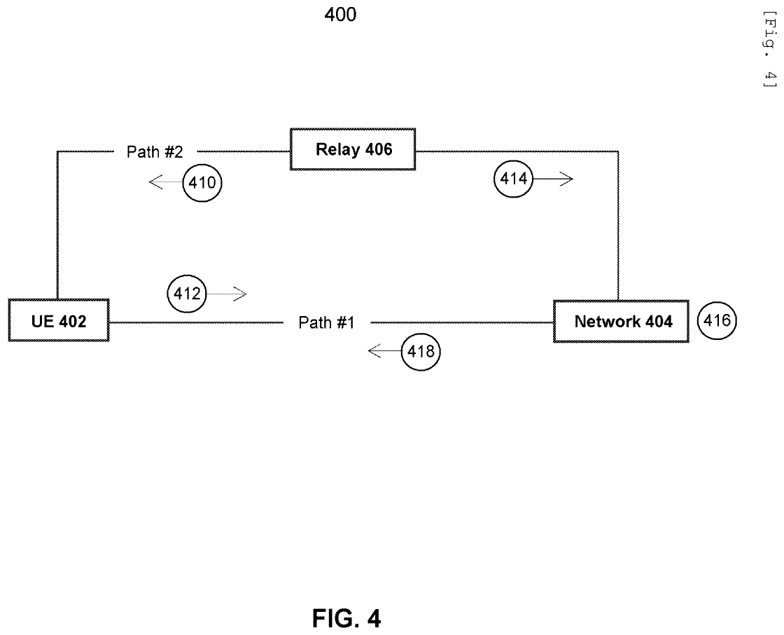

FIG. 4 is a schematic diagram illustrating a first approach for relay selection (or reselection), consistent with some embodiments of the present disclosure. Referring to FIG. 4, a communication system 400 includes a UE 402, a network 404, and a relay 406. The UE 402, the network 404, and the relay 406 are similar to the UE 302, the network 304, and the relay 306 of FIG. 3, respectively. For the sake of brevity, detailed descriptions of the UE 402, the network 404, and the relay 406 are omitted here. The first approach is also called a “UE-find-relay” approach. In the first approach, as shown in FIG. 4, at a step 410, the UE 402 receives a relay selection response message from the relay 406. In some embodiments, the UE 402 may first transmit a relay selection request message to the relay 406, and in response, receives the relay selection response message. The term “message” used in this disclosure can be anything conveyed in a communication, for example, information, signal(s), or data. Also, “a message” used in this disclosure can be a single message or multiple messages. In some embodiments, at least one of the relay selection request message or the relay selection response message is a discovery message transmitted during a device-to-device (D2D) discovery procedure between the UE 402 and the relay 406. In some embodiments, the relay selection response message may include at least one of: (1) one or more link quality parameters for the link between the UE 402 and the relay 406, (2) one or more attachment status parameters indicating attachment of the relay 406 to the network 404, or (3) one or more availability status parameters indicating availability of the relay 406 for being a relay for the UE 402. In some embodiments, the one or more link quality parameters for the link between the UE 402 and the relay 406 may include at least one of: one or more sidelink-reference signal received power (SL-RSRP) values measured on one or more sidelink signals transmitted between the UE 402 and the relay 406, or one or more sidelink discovery-reference signal received power (SD-RSRP) values measured on the one or more discovery messages during the discovery procedure.

At a step 412, the UE 402 transmits the relay selection response message to the network 404 so that the network 404 has knowledge about the characteristics of the link between the UE 402 and the relay 406.

At a step 414, the relay 406 transmits a measurement message to the network 404. The measurement message may be one or more measurement reports for one or more measurements performed by the relay 406 on the signals (e.g., downlink signals) transmitted between the network 404 and the relay 406. In some embodiments, the one or more measurements includes at least one of: (1) one or more link quality parameters for the link between the relay 406 and the network 404, or (2) one or more availability status parameters indicating availability of the relay 406 for a multi-path transmission. In some embodiments, the one or more link quality parameters for the link between the relay 406 and the network 404 may include at least one of: one or more reference signal received power (RSRP) values measured on one or more signals transmitted between the relay 406 and the network 404, or one or more reference signal received quality (RSRQ) values measured on the one or more signals transmitted between the relay 406 and the network 404.

At a step 416, the network 404 determines whether the relay 406 satisfies relay selection criteria based on at least one of: the relay selection response message received from the UE 402, or the measurement message received from the relay 406. In some embodiments, the relay selection criteria may include at least one of: (1) the one or more SL-RSRP values measured on the one or more sidelink signals transmitted between the UE 402 and the relay 404 exceed an SL-RSRP threshold, (2) the one or more SD-RSRP values measured on the one or more discovery messages exceed a SD-RSRP threshold, (3) the RSRP values measured on the one or more signals transmitted between the relay 406 and the network 404 exceed an RSRP threshold, or (4) the RSRQ values measured on the one or more signals transmitted between the relay 406 and the network 404 exceed an RSRQ threshold.

At a step 418, based on the determination results at the step 416, the network 404 selects the relay 406 as a relay for the UE 402, and transmits the selection result to the UE 402. The UE 402 may then trigger a multi-path transmission using the relay 406 as an U2N relay. In this way, by providing, to the network 404, both the relay selection response message including the characteristics of the link between the UE 402 and the relay 406, and the measurement message including the characteristics of the link between the relay 406 and the network 404, accuracy and reliability of relay selection (or reselection) are ensured.

Although FIG. 4 only shows one relay, i.e., the relay 406, as an example candidate relay, the embodiments of the present disclosure are not so limited. In some embodiments, there are multiple candidate relays in the communication system and the network 404 may select one or more relays from the multiple candidate relays. In this case, the UE 402 may communicate with the network 404 using a multi-path transmission including one or more indirect paths via the selected one or more relays.

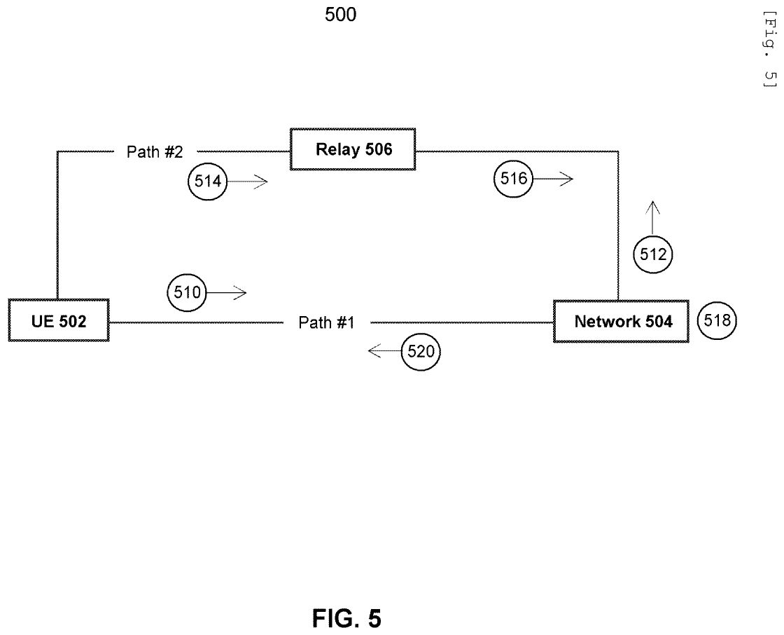

FIG. 5 is a schematic diagram illustrating a second approach for relay selection (or reselection), consistent with some embodiments of the present disclosure. Referring to FIG. 5, a communication system 500 includes a UE 502, a network 504, and a relay 506. The UE 502, the network 504, and the relay 506 are similar to the UE 302, the network 304, and the relay 306 of FIG. 3, respectively. For the sake of brevity, detailed descriptions of the UE 502, the network 504, and the relay 506 are omitted here. The second approach is also called a “Relay-find-UE” approach. In this approach, as shown in FIG. 5, at a step 510, the UE 502 transmits a relay selection request message to the network 504. And, at a step 512, the network 504 forwards the received relay selection request message to the relay 506. The relay selection request message may provide the information regarding the UE 502 or transmissions scheduled at the UE 502.

At a step 514, the relay 506 receives relay selection response message from the UE 502. In some embodiments, the relay selection response message is a discovery message transmitted during a D2D discovery procedure. In some embodiments, the relay selection response message may include one or more link quality parameters for the link between the UE 502 and the relay 506.

At a step 516, the relay 506 forwards the received relay selection response message to the network 504. In addition, the relay 506 also transmits a measurement message to the network 504. The measurement message may be one or more measurement reports for the measurements performed by the relay 506 on the signals (e.g., downlink signals) transmitted between the relay 506 and the network 504. In some embodiments, the one or more measurements include at least one of: (1) one or more link quality parameters for the link between the relay 506 and the network 504, or (2) one or more availability status parameters indicating availability of the relay 506 for being a relay in a multi-path transmission. In some embodiments, the one or more link quality parameters for the link between the relay 506 and the network 504 may include at least one of: one or more RSRP values measured on one or more signals transmitted between the relay 506 and the network 504, or one or more RSRQ values measured on the one or more signals transmitted between the relay 506 and the network 504.

At a step 518, the network 504 determines whether the relay 506 satisfies relay selection criteria based on at least one of: the relay selection response message, or the measurement message received from the relay 506. In some embodiments, the relay selection criteria may include at least one of: (1) the one or more SL-RSRP values measured on the one or more sidelink signals transmitted between the UE 502 and the relay 504 exceed an SL-RSRP threshold, (2) the one or more SD-RSRP values measured on the one or more discovery messages exceed a SD-RSRP threshold, (3) the RSRP values measured on the one or more signals transmitted between the relay 506 and the network 504 exceed an RSRP threshold, or (4) the RSRQ values measured on the one or more signals transmitted between the relay 506 and the network 504 exceed an RSRQ threshold.

At a step 520, based on the determination results at the step 518, the network 504 may select the relay 506 as a relay for the UE 502, and transmit the selection result to the UE 502. The UE 502 may then trigger a multi-path transmission using the relay 506 as an U2N relay. In this way, by providing, to the network 504, both the relay selection response message including the characteristics of the link between the UE 502 and the relay 506, and the measurement message including the characteristics of the link between the relay 506 and the network 504, accuracy and reliability of relay selection (or reselection) are ensured.

Although FIG. 5 only shows one relay, i.e., the relay 506, as an example candidate relay, the embodiments of the present disclosure are not so limited. In some embodiments, there are multiple candidate relays in the communication system and the network 504 may select one or more relays from the multiple candidate relays. In this case, the UE 502 may communicate with the network 504 using a multi-path transmission including one or more indirect paths via the selected one or more relays.

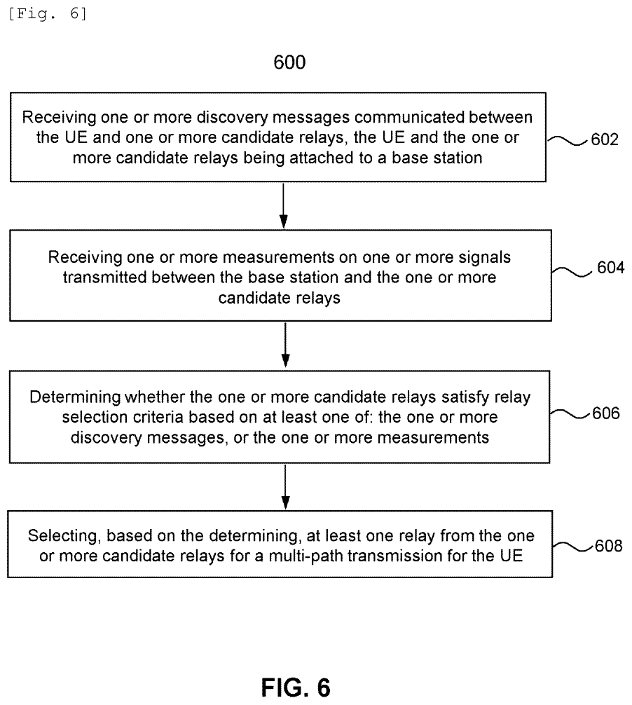

FIG. 6 is a schematic diagram illustrating a method for a base station for a multi-path communication, consistent with some embodiments of the present disclosure. The base station can be any base station currently existing (e.g., eNB, gNB) for LTE or NR, or a base station for a future generation (6G or any other future generation) RAT. For example, the base station can be the network 404 of FIG. 4 or the network 504 of FIG. 5. Referring to FIG. 6, a method 600 includes a step 602 of receiving, by a base station, one or more discovery messages communicated between the UE and one or more candidate relays, the UE and the one or more candidate relays being attached to the base station. The one or more candidate relays may include at least one of: one or more UEs that are different from the UE, or one or more road side units attached to the base station. The one or more discovery messages may include at least one of: (1) one or more relay selection requests, or (2) one or more relay selection response messages indicating one or more responses to the one or more relay selection requests. The one or more discovery messages may include at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission. The one or more link quality parameters for the one or more links between the UE and the one or more candidate relays may include at least one of: one or more SL-RSRP values measured on one or more sidelink signals transmitted between the UE and the one or more candidate relays, or one or more SD-RSRP values measured on the one or more discovery messages.

In a first embodiment, the base station is a base station such as the network 404 of FIG. 4. The UE and the one or more candidate relays are attached to the base station, for example, via Uu interface. In this embodiment, the base station receives, from the UE, one or more discovery messages (e.g., a relay selection response message) communicated between the UE and the one or more candidate relays. In this embodiment, the one or more discovery messages are obtained by the UE from the one or more candidate relays and forwarded to the base station. The one or more discovery messages may be one or more selection response messages and may include at least one of: (1) one or more link quality parameters for one or more links between the UE and the one or more candidate relays, or (2) one or more attachment status parameters indicating attachment of the one or more candidate relays to the base station.

In a second embodiment, the base station is a base station such as the network 504 of FIG. 5. In this embodiment, the base station receives, from the one or more relays, one or more discovery messages (e.g., a relay selection response message) communicated between the UE and the one or more candidate relays. In this embodiment, the one or more discovery messages are obtained by the one or more candidate relays from the UE and forwarded to the base station. The one or more discovery messages may be one or more selection response messages and may include one or more link quality parameters for one or more links between the UE and the one or more candidate relays.

The method 600 includes a step 604 of receiving, by the base station, one or more measurements on one or more signals transmitted between the base station and the one or more candidate relays. For example, in the first embodiment (e.g., FIG. 4), the base station receives one or more measurements on one or more signals transmitted between the base station and the one or more candidate relays. In the second embodiment (e.g., FIG. 5), the base station also receives one or more measurements on one or more signals transmitted between the base station and the one or more candidate relays. The one or more measurements may include at least one of: (1) one or more link quality parameters for one or more links between the base station the one or more candidate relays, or (2) one or more availability status parameters indicating availability of the one or more candidate relays for the multi-path transmission.

The method 600 includes a step 606 of determining, by the base station, whether the one or more candidate relays satisfy relay selection criteria based on at least one of: the one or more discovery messages, or the one or more measurements. For example, in the first embodiment (e.g., FIG. 4), the base station determines whether the one or more candidate relays satisfy relay selection criteria, based on at least one of: the one or more discovery messages received from the UE, or the one or more measurements received from the one or more candidate relays. In the second embodiment (e.g., FIG. 5), the base station determines whether the one or more candidate relays satisfy relay selection criteria, based on at least one of: the one or more discovery messages, or the one or more measurements received from the one or more candidate relays.

The method 600 includes a step 608 of selecting, by the base station and based on the determining, at least one relay from the one or more candidate relays for a multi-path transmission for the UE. In some embodiments, the base station may transmit the selection results to the UE so that the UE can trigger a multi-path transmission. The multi-path transmission may include a direct path between the UE and the base station, and an indirect path between the UE and the base station via the selected at least one relay. The relay selection criteria may include at least one of: (1) the one or more SL-RSRP values measured on the one or more sidelink signals transmitted between the UE and the one or more candidate relays exceed an SL-RSRP threshold, or (2) the one or more SD-RSRP values measured on the one or more discovery messages exceed a SD-RSRP threshold. Additionally, or alternatively, the relay selection criteria may include at least one of: (1) the RSRP values measured on the one or more signals transmitted between the base station and the one or more candidate relays exceed an RSRP threshold, or (2) the RSRQ values measured on the one or more signals transmitted between the base station and the one or more candidate relays exceed an RSRQ threshold.

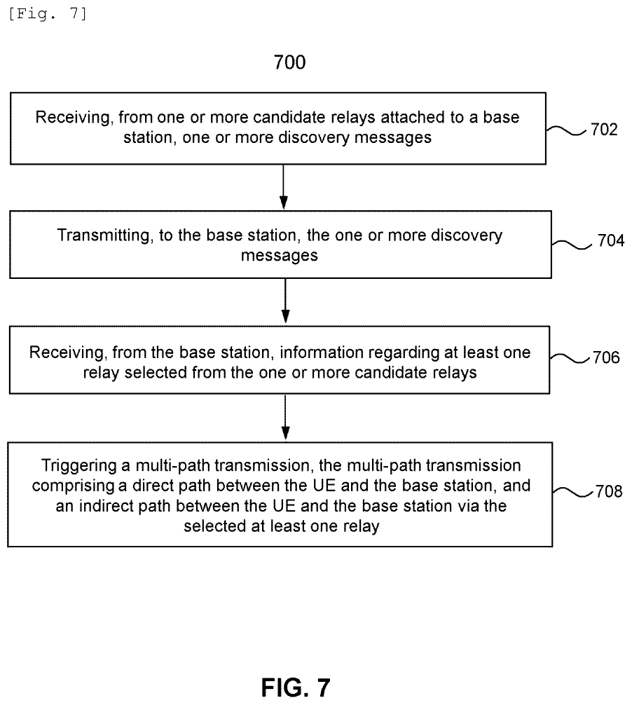

FIG. 7 is a schematic diagram illustrating a method for a UE for a multi-path communication, consistent with some embodiments of the present disclosure. For example, the UE, such as the UE 402 of FIG. 4, is attached to a base station. Referring to FIG. 7, a method 700 includes a step 702 of receiving, from one or more candidate relays attached to the base station, one or more discovery messages. The one or more candidate relays may be at least one of: one or more UEs that are different from the UE, or one or more road side units attached to the base station. In some embodiments, the one or more discovery messages may include at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission. In some embodiments, the one or more link quality parameters for the one or more links between the UE and the one or more candidate relays may include at least one of: one or more SL-RSRP values measured on one or more sidelink signals transmitted between the UE and the one or more candidate relays, or one or more SD-RSRP values measured on the one or more discovery messages.

The method 700 includes a step 704 of transmitting, to the base station, the one or more discovery messages. For example, the UE transmits the one or more discovery message to the base station so that the base station can have a knowledge about the characteristics of the link between the UE and the one or more candidate relays.

The method 700 includes a step 706 of receiving, from the base station, information regarding at least one relay selected from the one or more candidate relays. For example, the base station selects at least one relay from the one or more candidate relays, based on at least one of: the characteristics of the link between the UE and the one or more candidate relays received from the UE, or the information regarding the link between the base station and the one or more candidate relays, and sends the information about the selected at least one relay to the UE.

The method 700 includes a step 708 of triggering a multi-path transmission. For example, the UE may trigger a multi-path transmission using the selected at least one relay. The multi-path transmission may include a direct path between the UE and the base station, and an indirect path between the UE and the base station via the selected at least one relay.

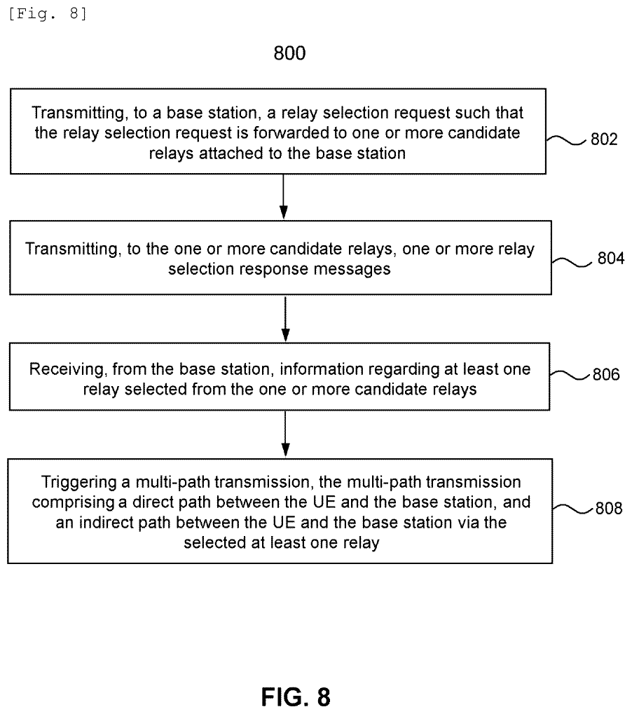

FIG. 8 is a schematic diagram illustrating a method for a UE for a multi-path communication, consistent with some embodiments of the present disclosure. For example, the UE, such as the UE 502 of FIG. 5, is attached to a base station. Referring to FIG. 8, a method 800 includes a step 802 of transmitting, to the base station, a relay selection request such that the relay selection request is forwarded to one or more candidate relays attached to the base station. The one or more candidate relays may be one or more UEs that are different from the UE, or one or more road side units that are attached to the base station. For example, in some embodiments, the UE may transmit the relay selection request to the base station such that the relay selection request can be forwarded to one or more candidate relays attached to the base station. The relay selection request may include the information regarding the UE or any scheduled transmissions from the UE.

The method 800 includes a step 804 of transmitting, to the one or more candidate relays, one or more relay selection response messages. For example, after transmitting the relay selection request, the UE may transmit one or more relay selection response messages, for example, during a D2D discovery procedure. The one or more relay selection response messages may include one or more link quality parameters for one or more links between the UE and the one or more candidate relays. The one or more link quality parameters for the one or more links between the UE and the one or more candidate relays may include at least one of: one or more SL-RSRP values measured on one or more sidelink signals transmitted between the UE and the one or more candidate relays, or one or more SD-RSRP values measured on the one or more discovery messages. The one or more candidate relays may further transmit the received one or more relay selection response messages to the base station, along with measurements performed on the signals transmitted between the one or more candidate relays and the base station.

The method 800 includes a step 806 of receiving, from the base station, information regarding at least one relay selected from the one or more candidate relays. For example, upon reception of the one or more relay selection response messages and the measurements results, the base station selects at least one relay from the one or more candidate relays and sends information regarding the selected at least one relay to the UE.

The method 800 includes a step 808 of triggering a multi-path transmission based on the selected at least one relay. The multi-path transmission may include a direct path between the UE and the base station, and an indirect path between the UE and the base station via the selected at least one relay.

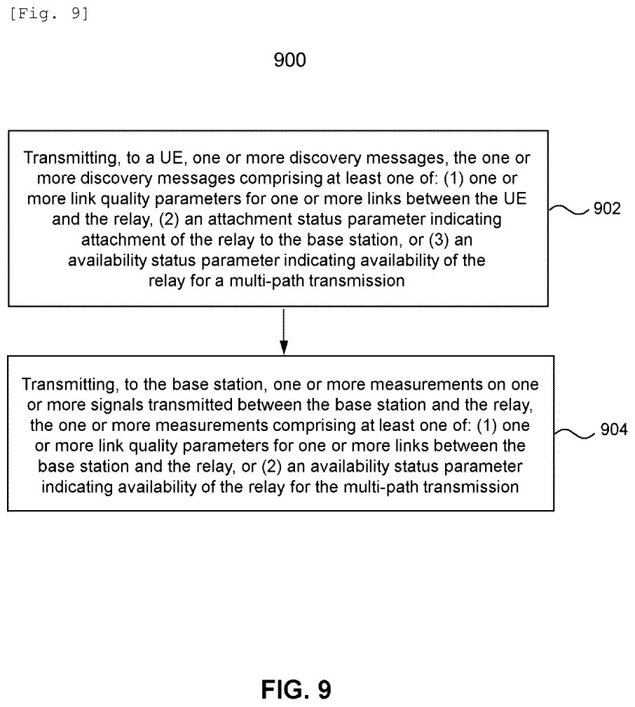

FIG. 9 is a schematic diagram illustrating a method for a relay for a multi-path communication, consistent with some embodiments of the present disclosure. The relay can be a candidate U2N relay for a UE in a communication. The relay and the UE may be attached to the same base station. For example, the relay (e.g., the relay 406 of FIG. 4) is a candidate relay for a UE (e.g., the UE 402 of FIG. 4) attached to a base station. Referring to FIG. 9, a method 900 includes a step 902 of transmitting, to the UE, one or more discovery messages. The one or more discovery messages may include at least one of: (1) one or more link quality parameters for one or more links between the UE and the relay, (2) an attachment status parameter indicating attachment of the relay to the base station, or (3) an availability status parameter indicating availability of the relay for multi-path transmission. The UE may further transmit the received one or more discovery messages to the base station. The one or more link quality parameters for the one or more links between the UE and the one or more candidate relays may include at least one of: one or more SL-RSRP values measured on one or more sidelink signals transmitted between the UE and the one or more candidate relays, or one or more SD-RSRP values measured on the one or more discovery messages.

The method 900 include a step 904 of transmitting, to the base station, one or more measurements on one or more signals transmitted between the base station and the relay. The one or more measurements may include at least one of: (1) one or more link quality parameters for one or more links between the base station and the relay, or (2) an availability status parameter indicating availability of the relay for the multi-path transmission. In some embodiments, the one or more link quality parameters for the one or more links between the base station and the relay may include at least one of: one or more RSRP values measured on one or more signals transmitted between the base station and the relay, or one or more RSRQ values measured on the one or more signals transmitted between the base station and the relay. Based on at least one of: the one or more discovery messages received from the UE, or the one or more measurements received from the relay, the base station may determine whether the relay is a suitable relay for the UE.

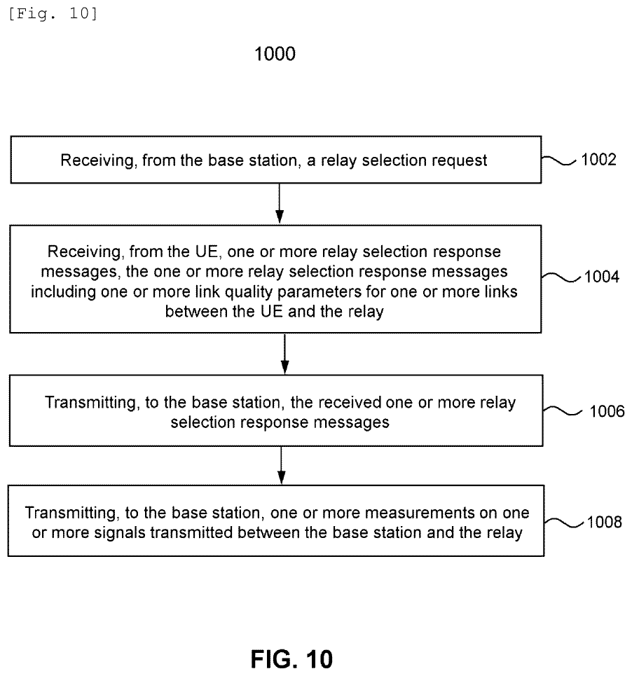

FIG. 10 is a schematic diagram illustrating a method for a relay for a multi-path communication, consistent with some embodiments of the present disclosure. The relay can be a candidate U2N relay for a UE in a communication. The relay and the UE may be attached to the same base station. For example, the relay (e.g., the relay 506 of FIG. 5) is a candidate relay for a UE (e.g., the UE 502 of FIG. 5) attached to a base station. Referring to FIG. 10, a method 1000 includes a step 1002 of receiving, from the base station, a relay selection request. For example, in some embodiments, the base station may receive the relay selection request from the UE and forward it to the relay. The relay selection request may include the information regarding the UE or any scheduled transmissions from the UE.

The method 1000 include a step 1004 of receiving, from the UE, one or more relay selection response messages, the one or more relay selection response messages including one or more link quality parameters for one or more links between the UE and the relay. For example, the relay may receive the one or more relay selection response messages from the UE during a D2D discovery procedure. The one or more relay selection response messages may include one or more link quality parameters for the link between the UE and the relay. The one or more link quality parameters may include at least one of: one or more SL-RSRP values measured on one or more sidelink signals transmitted between the UE and the relay, or one or more SD-RSRP values measured on the one or more discovery messages.

The method 1000 includes a step 1006 of transmitting, to the base station, the received one or more relay selection response messages. For example, the relay may further transmit the received one or more relay selection response messages to the base station.

The method 1000 includes a step 1008 of transmitting, to the base station, one or more measurements on one or more signals transmitted between the base station and the relay. The one or more measurements may include at least one of: (1) one or more link quality parameters for one or more links between the base station and the relay, or (2) one or more availability status parameters indicating availability of the relay for a multi-path transmission. Based on at least one of: the one or more relay selection response messages, or the one or more measurements received from the relay, the base station may determine whether the relay is a suitable relay for the UE.

The methods described in this disclosure can be applied to any communications, for example, LTE or NR or a future generation (e.g., 6G or any future generation) communications. The methods described in this disclosure can also be applied to a communication involving any number of UEs. The methods described in this disclosure can also be applied to other systems, for example, the systems that comply with other standards (e.g., the Institute of Electrical and Electronics Engineers (IEEE) standards), for example, IEEE 802.11 technologies.

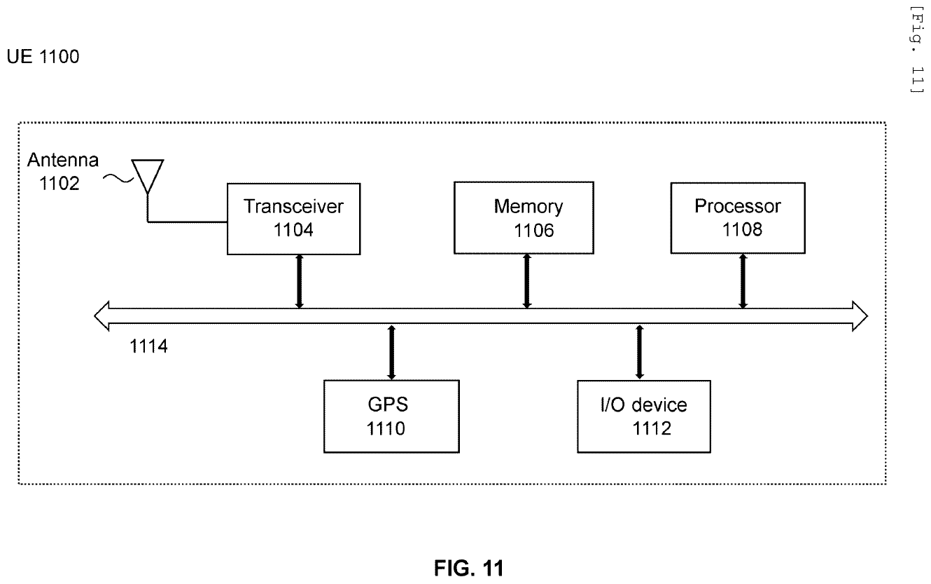

FIG. 11 is a block diagram of a UE 1100, consistent with some embodiments of the present disclosure. For example, the UE 1100 may be the UE 402 of FIG. 4 or the UE 502 of FIG. 5 that triggers a multi-path transmission. Alternatively, the UE 1100 may be a relay UE (e.g., a U2N relay) that performs relay function for another UE. For example, the UE 1100 may be the relay 406 of FIG. 4 or the relay 506 of FIG. 5. The UE 1100 may be mounted in a moving vehicle or in a fixed position. UE 1100 may take any form, including but not limited to, a vehicle, a component mounted in a vehicle, a road-side unit, a laptop computer, a wireless terminal including a mobile phone, a wireless handheld device, or wireless personal device, or any other form.

Referring to FIG. 11, the UE 1100 may include antenna 1102 that may be used for transmission or reception of electromagnetic signals to/from a base station or other UEs. The antenna 1102 may include one or more antenna elements and may enable different input-output antenna configurations, for example, multiple input multiple output (MIMO) configuration, multiple input single output (MISO) configuration, and single input multiple output (SIMO) configuration. In some embodiments, the antenna 1102 may include multiple (e.g., tens or hundreds) antenna elements and may enable multi-antenna functions such as beamforming. In some embodiments, the antenna 1102 is a single antenna.

The UE 1100 may include a transceiver 1104 that is coupled to the antenna 1102. The transceiver 1104 may be a wireless transceiver at the UE 1100 and may communicate bi-directionally with a base station or other UEs. For example, the transceiver 1104 may receive/transmit wireless signals from/to a base station via downlink/uplink communication. The transceiver 1104 may also receive/transmit wireless signals from/to another UE or road side unit via sidelink communication. The transceiver 1104 may include a modem to modulate the packets and provide the modulated packets to the antenna 1102 for transmission, and to demodulate packets received from the antenna 1102.

The UE 1100 may include a memory 1106. The memory 1106 may be any type of computer-readable storage medium including volatile or non-volatile memory devices, or a combination thereof. The computer-readable storage medium includes, but is not limited to, non-transitory computer storage media. A non-transitory storage medium may be accessed by a general purpose or special purpose computer. Examples of non-transitory storage medium include, but are not limited to, a portable computer diskette, a hard disk, random access memory (RAM), read-only memory (ROM), an erasable programmable read-only memory (EPROM), electrically erasable programmable ROM (EEPROM), a digital versatile disk (DVD), flash memory, compact disk (CD) ROM or other optical disk storage, magnetic disk storage or other magnetic storage devices, etc. A non-transitory medium may be used to carry or store desired program code means (e.g., instructions and/or data structures) and may be accessed by a general-purpose or special-purpose computer, or a general-purpose or special-purpose processor. In some examples, the software/program code may be transmitted from a remote source (e.g., a website, a server, etc.) using a coaxial cable, fiber optic cable, twisted pair, digital subscriber line (DSL), or wireless technologies such as infrared, radio, and microwave. In such examples, the coaxial cable, fiber optic cable, twisted pair, DSL, or wireless technologies such as infrared, radio, and microwave are within the scope of the definition of medium. Combinations of the above examples are also within the scope of computer-readable medium.

The memory 1106 may store information related to identities of the UE 1100 and the signals and/or data received by antenna 1102. The memory 1106 may also store post-processing signals and/or data. The memory 1106 may also store computer-readable program instructions, mathematical models, and algorithms that are used in signal processing in receiver 1104 and computations in processor 1108. The memory 1106 may further store computer-readable program instructions for execution by the processor 1108 to the operate UE 1100 to perform various functions described in this disclosure. In some examples, the memory 1106 may include a basic input/output system (BIOS) which may control basic hardware or software operation such as the interaction with peripheral components or devices.

The computer-readable program instructions of the present disclosure may be assembler instructions, instruction-set-architecture (ISA) instructions, machine instructions, machine-dependent instructions, microcode, firmware instructions, state-setting data, or source code or object code written in any combination of one or more programming languages, including an object-oriented programming language, and conventional procedural programming languages. The computer-readable program instructions may execute entirely on a computing device as a stand-alone software package, or partly on a first computing device and partly on a second computing device remote from the first computing device. In the latter scenario, the second, remote computing device may be connected to the first computing device through any type of network, including a local area network (LAN) or a wide area network (WAN).

The UE 1100 may include a processor 1108 that may include a hardware device with processing capabilities. The processor 1108 may include at least one of a general-purpose processor, a digital signal processor (DSP), a central processing unit (CPU), a microcontroller, an application specific integrated circuit (ASIC), a field programmable gate array (FPGA), a programmable logic device, a discrete gate or transistor logic component, a discrete hardware component, or other programmable logic device. Examples of the general-purpose processor include, but are not limited to, a microprocessor, any conventional processor, a controller, a microcontroller, or a state machine. In some embodiments, the processor 1108 may be implemented using a combination of devices (e.g., a combination of a DSP and a microprocessor, multiple microprocessors, one or more microprocessors in conjunction with a DSP core, or any other such configuration). The processor 1108 may receive, from transceiver 1104, downlink signals or sidelink signals and further process the signals. The processor 1108 may also receive, from the transceiver 1104, data packets and further process the packets. In some embodiments, the processor 1108 may be configured to operate a memory using a memory controller. In some embodiments, a memory controller may be integrated into the processor 1108. The processor 1108 may be configured to execute computer-readable instructions stored in a memory (e.g., the memory 1106) to cause the UE 1100 to perform various functions.

The UE 1100 may include a global positioning system (GPS) 1110. The GPS 1110 may be used for enabling location-based services or other services based on a geographical position of the UE 1100 and/or synchronization among UEs. The GPS 1110 may receive global navigation satellite systems (GNSS) signals from a single satellite or a plurality of satellite signals via the antenna 1102 and provide a geographical position of the UE 1100 (e.g., coordinates of the UE 1100). In some embodiments, the GPS 1110 is omitted. In some embodiments, a timer is included.

The UE 1100 may include an input/output (I/O) device 1112 that may be used to communicate a result of signal processing and computation to a user or another device. The I/O device 1112 may include a user interface including a display and an input device to transmit a user command to the processor 1108. The display may be configured to display a status of signal reception at the UE 1100, the data stored at memory 1106, a status of signal processing, and a result of computation, etc. The display may include, but is not limited to, a cathode ray tube (CRT), a liquid crystal display (LCD), a light-emitting diode (LED), a gas plasma display, a touch screen, or other image projection devices for displaying information to a user. The input device may be any type of computer hardware equipment used to receive data and control signals from a user. The input device may include, but is not limited to, a keyboard, a mouse, a scanner, a digital camera, a joystick, a trackball, cursor direction keys, a touchscreen monitor, or audio/video commanders, etc.

The UE 1100 may further include a machine interface 1114, such as an electrical bus that connects the transceiver 1104, the memory 1106, the processor 1108, the GPS 1110, and the I/O device 1112.

In some embodiments, the UE 1100 may be a UE that plans to perform a multi-path transmission with a base station in a communication. The processor 1108 may be configured or programmed to execute the instructions stored in the memory 1106 to receive, from one or more candidate relays attached to the base station, one or more discovery messages, the one or more discovery messages including at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission; transmit, to the base station, the one or more discovery messages; receive, from the base station, information regarding at least one relay selected from the one or more candidate relays; and trigger a multi-path transmission, the multi-path transmission including a direct path between the UE and the base station, and an indirect path between the UE and the base station via the selected at least one relay.

In some embodiments, the UE 1100 may be a UE that plans to perform a multi-path transmission with a base station in a communication. The processor 1108 may be configured or programmed to execute the instructions stored in the memory 1106 to transmit, to the base station, a relay selection request such that the relay selection request is forwarded to one or more candidate relays attached to the base station; transmit, to the one or more candidate relays, one or more relay selection response messages, the one or more relay selection response messages including one or more link quality parameters for one or more links between the UE and the one or more candidate relays; receive, from the base station, information regarding at least one relay selected from the one or more candidate relays; and trigger a multi-path transmission, the multi-path transmission comprising a direct path between the UE and the base station, and an indirect path between the UE and the base station via the selected at least one relay.

In some embodiments, the UE 1100 may be a candidate relay UE for another UE that plans to perform a multi-path transmission with a base station in a communication. The processor 1108 may be configured or programmed to execute the instructions stored in the memory 1106 to transmit, to the UE, one or more discovery messages, the one or more discovery messages comprising at least one of: (1) one or more link quality parameters for one or more links between the UE and the relay, (2) an attachment status parameter indicating attachment of the relay to the base station, or (3) an availability status parameter indicating availability of the relay for multi-path transmission; and transmit, to the base station, one or more measurements on one or more signals transmitted between the base station and the relay, the one or more measurements comprising at least one of: (1) one or more link quality parameters for one or more links between the base station and the relay, or (2) an availability status parameter indicating availability of the relay for the multi-path transmission.

In some embodiments, the UE 1100 may be a candidate relay UE for another UE that plans to perform a multi-path transmission with a base station in a communication. The processor 1108 may be configured or programmed to execute the instructions stored in the memory 1106 to receive, from the base station, a relay selection request; receive, from the UE, one or more relay selection response messages, the one or more relay selection response messages including one or more link quality parameters for one or more links between the UE and the relay; transmit, to the base station, the received one or more relay selection response messages; and transmit, to the base station, one or more measurements on one or more signals transmitted between the base station and the relay, the one or more measurements comprising at least one of: (1) one or more link quality parameters for one or more links between the base station and the relay, or (2) one or more availability status parameters indicating availability of the relay for a multi-path transmission.

As used in this disclosure, use of the term “or” in a list of items indicates an inclusive list. The list of items may be prefaced by a phrase such as “at least one of” or “one or more of.” For example, a list of at least one of A, B, or C includes A or B or C or AB (i.e., A and B) or AC or BC or ABC (i.e., A and B and C). Also, as used in this disclosure, prefacing a list of conditions with the phrase “based on” shall not be construed as “based only on” the set of conditions and rather shall be construed as “based at least in part on” the set of conditions. For example, an outcome described as “based on condition A” may be based on both a condition A and a condition B without departing from the scope of this disclosure.

In this specification, the terms “comprise,” “include,” or “contain” may be used interchangeably and have the same meaning and are to be construed as inclusive and open-ended. The terms “comprise,” “include,” or “contain” may be used before a list of elements and indicate that at least all of the listed elements within the list exist but other elements that are not in the list may also be present. For example, if A comprises B and C, both {B, C} and {B, C, D} are within the scope of A.

The present disclosure, in connection with the accompanied drawings, describes example configurations that are not representative of all the examples that may be implemented or all configurations that are within the scope of this disclosure. The term “exemplary” should not be construed as “preferred” or “advantageous compared to other examples” but rather “an illustration, an instance or an example.” By reading this disclosure, including the description of the embodiments and the drawings, it will be appreciated by a person of ordinary skills in the art that the technology disclosed herein may be implemented using alternative embodiments. The person of ordinary skill in the art would appreciate that the embodiments, or certain features of the embodiments described herein, may be combined to arrive at yet other embodiments for practicing the technology described in the present disclosure. Thus, the disclosure is not limited to the examples and designs described herein but is to be accorded the broadest scope consistent with the principles and novel features disclosed herein.

The flowcharts and block diagrams in the figures illustrate examples of the architecture, functionality, and operation of possible implementations of systems, methods, and devices according to various embodiments. It should be noted that, in some alternative implementations, the functions noted in blocks may occur out of the order noted in the figures. For example, two blocks shown in succession may, in fact, be executed substantially concurrently, or the blocks may sometimes be executed in the reverse order, depending upon the functionality involved. Likewise, additional steps may be included in such methods, and certain steps may be omitted or combined, in methods consistent with various embodiments.

It is understood that the described embodiments are not mutually exclusive, and elements, components, materials, or steps described in connection with one example embodiment may be combined with, or eliminated from, other embodiments in suitable ways to accomplish desired design objectives.

Reference herein to “some embodiments” or “some exemplary embodiments” means that a particular feature, structure, or characteristic described in connection with the embodiment can be included in at least one embodiment. The appearance of the phrases “one embodiment” “some embodiments” or “another embodiment” in various places in the present disclosure do not all necessarily refer to the same embodiment, nor are separate or alternative embodiments necessarily mutually exclusive of other embodiments.

Additionally, the articles “a” and “an” as used in the present disclosure and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

Although the elements in the following method claims, if any, are recited in a particular sequence, unless the claim recitations otherwise imply a particular sequence for implementing some or all of those elements, those elements are not necessarily intended to be limited to being implemented in that particular sequence.

It is appreciated that certain features of the present disclosure, which are, for clarity, described in the context of separate embodiments, may also be provided in combination in a single embodiment. Conversely, various features of the specification, which are, for brevity, described in the context of a single embodiment, may also be provided separately or in any suitable subcombination or as suitable in any other described embodiment of the specification. Certain features described in the context of various embodiments are not essential features of those embodiments, unless noted as such.

It will be further understood that various modifications, alternatives, and variations in the details, materials, and arrangements of the parts which have been described and illustrated in order to explain the nature of described embodiments may be made by those skilled in the art without departing from the scope. Accordingly, the following claims embrace all such alternatives, modifications, and variations that fall within the terms of the claims.

Clause 1: A method for selecting at least one relay for a user equipment (UE) in a communication system, the method comprising:
receiving, by a base station, one or more discovery messages communicated between the UE and one or more candidate relays, the UE and the one or more candidate relays being attached to the base station; receiving, by the base station, one or more measurements on one or more signals transmitted between the base station and the one or more candidate relays;
determining, by the base station, whether the one or more candidate relays satisfy relay selection criteria based on at least one of: the one or more discovery messages, or the one or more measurements; and
selecting, by the base station and based on the determining, at least one relay from the one or more candidate relays for a multi-path transmission for the UE.

Clause 2: The method of clause 1, wherein the one or more candidate relays comprise at least one of: one or more UEs that are different from the UE, or one or more road side units attached to the base station.

Clause 3: The method of clause 1, wherein the one or more discovery messages comprise at least one of: (1) one or more relay selection requests, or (2) one or more relay selection response messages indicating one or more responses to the one or more relay selection requests.

Clause 4: The method of clause 1, wherein the one or more discovery messages comprise at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission.

Clause 5: The method of clause 4, wherein the one or more link quality parameters for the one or more links between the UE and the one or more candidate relays comprise at least one of: one or more sidelink-reference signal received power (SL-RSRP) values measured on one or more sidelink signals transmitted between the UE and the one or more candidate relays, or one or more sidelink discovery-reference signal received power (SD-RSRP) values measured on the one or more discovery messages.

Clause 6: The method of clause 5, wherein the relay selection criteria comprise at least one of: (1) the one or more SL-RSRP values measured on the one or more sidelink signals transmitted between the UE and the one or more candidate relays exceed an SL-RSRP threshold, or (2) the one or more SD-RSRP values measured on the one or more discovery messages exceed a SD-RSRP threshold.

Clause 7: The method of clause 1, wherein the one or more measurements comprises at least one of: (1) one or more link quality parameters for one or more links between the base station the one or more candidate relays, or (2) one or more availability status parameters indicating availability of the one or more candidate relays for the multi-path transmission.

Clause 8: The method of clause 7, wherein the one or more link quality parameters for the one or more links between the base station and the one or more candidate relays comprise at least one of: one or more RSRP values measured on one or more signals transmitted between the base station and the one or more candidate relays, or one or more RSRQ values measured on the one or more signals transmitted between the base station and the one or more candidate relays.

Clause 9: The method of clause 8, wherein the relay selection criteria comprise at least one of: (1) the RSRP values measured on the one or more signals transmitted between the base station and the one or more candidate relays exceed an RSRP threshold, or (2) the RSRQ values measured on the one or more signals transmitted between the base station and the one or more candidate relays exceed an RSRQ threshold.

Clause 10: The method of clause 1, wherein receiving the one or more discovery messages communicated between the UE and the one or more candidate relays further comprises: receiving, from the UE, the one or more discovery messages that are obtained from the one or more candidate relays, the one or more discovery messages being one or more selection response messages and comprising at least one of: (1) one or more link quality parameters for one or more links between the UE and the one or more candidate relays, or (2) one or more attachment status parameters indicating attachment of the one or more candidate relays to the base station.

Clause 11: The method of clause 10, wherein receiving the one or more measurements on the one or more signals transmitted between the base station and the one or more candidate relays further comprises:
receiving, from the one or more candidate relays, the one or more measurements, the one or more measurements comprising at least one of: (1) one or more link quality parameters for one or more links between the base station the one or more candidate relays, or (2) one or more availability status parameters indicating availability of the one or more candidate relays for the multi-path transmission.

Clause 12: The method of clause 1, wherein receiving the one or more discovery messages communicated between the UE and the one or more candidate relays further comprises: receiving, from the one or more candidate relays, the one or more discovery messages that are obtained from the UE, the one or more discovery messages being one or more selection response messages and comprising one or more link quality parameters for one or more links between the UE and the one or more candidate relays.

Clause 13: The method of clause 12, wherein receiving the one or more discovery messages communicated between the UE and the one or more candidate relays further comprises: forwarding, to the one or more candidate relays, one or more relay selection requests received from the UE; and receiving, from the one or more candidate relays, one or more relay selection response messages obtained from the UE in response to the one or more relay selection requests.

Clause 14: The method of clause 12, wherein receiving the one or more measurements on the one or more signals transmitted between the base station and the one or more candidate relays further comprises:
receiving, from the one or more candidate relays, the one or more measurements, the one or more measurements comprising at least one of: one or more link quality parameters for one or more links between the base station the one or more candidate relays, or (2) one or more availability status parameters indicating availability of the one or more candidate relays for the multi-path transmission.

Clause 15: The method of clause 1, further comprising: transmitting, to the UE, the selected at least one relay to trigger the multi-path transmission at the UE, the multi-path transmission comprising a direct path between the UE and the base station, and an indirect path between the UE and the base station via the selected at least one relay.

Clause 16: A method for a user equipment (UE), the UE being attached to a base station in a communication system, the method comprising:
receiving, from one or more candidate relays attached to the base station, one or more discovery messages, the one or more discovery messages comprising at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission;
transmitting, to the base station, the one or more discovery messages;
receiving, from the base station, information regarding at least one relay selected from the one or more candidate relays; and
triggering a multi-path transmission, the multi-path transmission comprising a direct path between the UE and the base station, and an indirect path between the UE and the base station via the selected at least one relay.

Clause 17: The method of clause 16, wherein the one or more candidate relays comprise at least one of: one or more UEs that are different from the UE, or one or more road side units attached to the base station.

Clause 18: The method of clause 16, wherein the one or more link quality parameters for the one or more links between the UE and the one or more candidate relays comprise at least one of: one or more sidelink-reference signal received power (SL-RSRP) values measured on one or more sidelink signals transmitted between the UE and the one or more candidate relays, or one or more sidelink discovery-reference signal received power (SD-RSRP) values measured on the one or more discovery messages.

Clause 19: A method for a user equipment (UE), the UE being attached to a base station in a communication system, the method comprising:
transmitting, to the base station, a relay selection request such that the relay selection request is forwarded to one or more candidate relays attached to the base station;
transmitting, to the one or more candidate relays, one or more relay selection response messages, the one or more relay selection response messages comprising one or more link quality parameters for one or more links between the UE and the one or more candidate relays;
receiving, from the base station, information regarding at least one relay selected from the one or more candidate relays; and
triggering a multi-path transmission, the multi-path transmission comprising a direct path between the UE and the base station, and an indirect path between the UE and the base station via the selected at least one relay.

Clause 20: The method of clause 19, wherein the one or more candidate relays comprise at least one of: one or more UEs that are different from the UE, or one or more road side units that are attached to the base station.

Clause 21: The method of clause 19, wherein the one or more link quality parameters for the one or more links between the UE and the one or more candidate relays comprise at least one of: one or more sidelink-reference signal received power (SL-RSRP) values measured on one or more sidelink signals transmitted between the UE and the one or more candidate relays, or one or more sidelink discovery-reference signal received power (SD-RSRP) values measured on the one or more discovery messages.

Clause 22: A method for a relay for a user equipment (UE) in a communication, the relay and the UE being attached to a base station, the method comprising:
transmitting, to the UE, one or more discovery messages, the one or more discovery messages comprising at least one of: (1) one or more link quality parameters for one or more links between the UE and the relay, (2) an attachment status parameter indicating attachment of the relay to the base station, or (3) an availability status parameter indicating availability of the relay for multi-path transmission; and
transmitting, to the base station, one or more measurements on one or more signals transmitted between the base station and the relay, the one or more measurements comprising at least one of: (1) one or more link quality parameters for one or more links between the base station and the relay, or (2) an availability status parameter indicating availability of the relay for the multi-path transmission.

Clause 23: A method for a relay for a user equipment (UE) in a communication, the relay and the UE being attached to a base station, the method comprising: receiving, from the base station, a relay selection request;
receiving, from the UE, one or more relay selection response messages, the one or more relay selection response messages including one or more link quality parameters for one or more links between the UE and the relay;
transmitting, to the base station, the received one or more relay selection response messages; and transmitting, to the base station, one or more measurements on one or more signals transmitted between the base station and the relay, the one or more measurements comprising at least one of: (1) one or more link quality parameters for one or more links between the base station and the relay, or (2) one or more availability status parameters indicating availability of the relay for a multi-path transmission.

Clause 24: A base station in a communication system, the base station comprising:
a memory storing an instruction; and
a processor configured to execute the instruction stored in the memory to:
receive one or more discovery messages communicated between the UE and the one or more candidate relays, the UE and the one or more candidate relays being attached to the base station;
receive one or more measurements on one or more signals transmitted between the base station and the one or more candidate relays;
determine whether the one or more candidate relays satisfy relay selection criteria based on at least one of: the one or more discovery messages, or the one or more measurements; and
select at least one relay from the one or more candidate relays for a multi-path transmission for the UE.

Clause 25: The base station of clause 24, wherein the one or more candidate relays comprise at least one of: one or more UEs that are different from the UE, or one or more road side units attached to the base station.

Clause 26: The base station of clause 24, wherein the one or more discovery messages comprise at least one of: (1) one or more relay selection requests, or (2) one or more relay selection response messages indicating one or more responses to the one or more relay selection requests.

Clause 27: The base station of clause 24, wherein the one or more discovery messages comprise at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission.

Clause 28: The base station of clause 27, wherein the one or more link quality parameters for the one or more links between the UE and the one or more candidate relays comprise at least one of: one or more sidelink-reference signal received power (SL-RSRP) values measured on one or more signals transmitted between the UE and the one or more candidate relays, or one or more sidelink discovery-reference signal received power (SD-RSRP) values measured on the one or more discovery messages.

Clause 29: The base station of clause 28, wherein the relay selection criteria comprise at least one of: (1) the one or more SL-RSRP values measured on the one or more sidelink signals transmitted between the UE and the one or more candidate relays exceed an SL-RSRP threshold, or (2) the one or more SD-RSRP values measured on the one or more discovery messages exceed a SD-RSRP threshold.

Clause 30: The base station of clause 24, wherein the one or more measurements comprises at least one of: (1) one or more link quality parameters for one or more links between the base station the one or more candidate relays, or (2) one or more availability status parameters indicating availability of the one or more candidate relays for the multi-path transmission.

Clause 31: The base station of clause 30, wherein the one or more link quality parameters for the one or more links between the base station and the one or more candidate relays comprise at least one of: one or more RSRP values measured on one or more signals transmitted between the base station and the one or more candidate relays, or one or more RSRQ values measured on the one or more signals transmitted between the base station and the one or more candidate relays.

Clause 32: The base station of clause 31, wherein the relay selection criteria comprise at least one of: (1) the RSRP values measured on the one or more signals transmitted between the base station and the one or more candidate relays exceed an RSRP threshold, or (2) the RSRQ values measured on the one or more signals transmitted between the base station and the one or more candidate relays exceed an RSRQ threshold.

Clause 33: The base station of clause 24, wherein the processor is further configured to execute the instruction stored in the memory to:
receive, from the UE, the one or more discovery messages that are obtained from the one or more candidate relays, the one or more discovery messages being one or more selection response messages and comprising at least one of: (1) one or more link quality parameters for one or more links between the UE and the one or more candidate relays, or (2) one or more attachment status parameters indicating attachment of the one or more candidate relays to the base station.

Clause 34: The base station of clause 33, wherein the processor is further configured to execute the instruction stored in the memory to:
receive, from the one or more candidate relays, the one or more measurements, the one or more measurements comprising at least one of: (1) one or more link quality parameters for one or more links between the base station the one or more candidate relays, or (2) one or more availability status parameters indicating availability of the one or more candidate relays for the multi-path transmission.

Clause 35: The base station of clause 24, wherein the processor is further configured to execute the instruction stored in the memory to:
receive, from the one or more candidate relays, the one or more discovery messages that are obtained from the UE, the one or more discovery messages being one or more selection response messages and comprising one or more link quality parameters for one or more links between the UE and the one or more candidate relays.

Clause 36: The base station of clause 35, wherein the processor is further configured to execute the instruction stored in the memory to:
forward, to the one or more candidate relays, one or more relay selection requests received from the UE; and receive, from the one or more candidate relays, one or more relay selection response messages obtained from the UE in response to the one or more relay selection requests.

Clause 37: The base station of clause 35, wherein the processor is further configured to execute the instruction stored in the memory to:
receive, from the one or more candidate relays, the one or more measurements, the one or more measurements comprising at least one of: one or more link quality parameters for one or more links between the base station the one or more candidate relays, or (2) one or more availability status parameters indicating availability of the one or more candidate relays for the multi-path transmission.

Clause 38: The base station of clause 24, wherein the processor is further configured to execute the instruction stored in the memory to:
transmit, to the UE, the selected at least one relay to trigger the multi-path transmission at the UE, the multi-path transmission comprising a direct path between the UE and the base station, and an indirect path between the UE and the base station via the selected at least one relay.

Clause 39: A user equipment (UE) for a communication, the UE being attached to a base station, the UE comprising: a memory storing an instruction; and a processor configured to execute the instruction stored in the memory to:
receive, from one or more candidate relays attached to the base station, one or more discovery messages, the one or more discovery messages comprising at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission;
transmit, to the base station, the one or more discovery messages;
receive, from the base station, information regarding at least one relay selected from the one or more candidate relays; and
trigger a multi-path transmission, the multi-path transmission comprising a direct path between the UE and the base station, and an indirect path between the UE and the base station via the selected at least one relay.

Clause 40: The UE of clause 39, wherein the one or more candidate relays comprise at least one of: one or more UEs that are different from the UE, or one or more road side units attached to the base station.

Clause 41: The UE of clause 39, wherein the one or more link quality parameters for the one or more links between the UE and the one or more candidate relays comprise at least one of: one or more sidelink-reference signal received power (SL-RSRP) values measured on one or more signals transmitted between the UE and the one or more candidate relays, or one or more sidelink discovery-reference signal received power (SD-RSRP) values measured on the one or more discovery messages.

Clause 42: A user equipment (UE) for a communication, the UE being attached to a base station, the UE comprising: a memory storing an instruction; and a processor configured to execute the instruction stored in the memory to:
transmit, to the base station, a relay selection request such that the relay selection request is forwarded to one or more candidate relays attached to the base station; transmit, to the one or more candidate relays, one or more relay selection response messages, the one or more relay selection response messages comprising one or more link quality parameters for one or more links between the UE and the one or more candidate relays;
receive, from the base station, information regarding at least one relay selected from the one or more candidate relays; and
trigger a multi-path transmission, the multi-path transmission comprising a direct path between the UE and the base station, and an indirect path between the UE and the base station via the selected at least one relay.

Clause 43: The UE of clause 42, wherein the one or more candidate relays comprise at least one of: one or more UEs that are different from the UE, or one or more road side units that are attached to the base station.

Clause 44: The UE of clause 42, wherein the one or more link quality parameters for the one or more links between the UE and the one or more candidate relays comprise at least one of: one or more sidelink-reference signal received power (SL-RSRP) values measured on one or more sidelink signals transmitted between the UE and the one or more candidate relays, or one or more sidelink discovery-reference signal received power (SD-RSRP) values measured on the one or more discovery messages.

Clause 45: A relay for a user equipment (UE) in a communication, the relay and the UE being attached to a base station, the relay comprising:
a memory storing an instruction; and
a processor configured to execute the instruction stored in the memory to:
transmit, to the UE, one or more discovery messages, the one or more discovery messages comprising at least one of: (1) one or more link quality parameters for one or more links between the UE and the relay, (2) an attachment status parameter indicating attachment of the relay to the base station, or (3) an availability status parameter indicating availability of the relay for multi-path transmission; and
transmit, to the base station, one or more measurements on one or more signals transmitted between the base station and the relay, the one or more measurements comprising at least one of: (1) one or more link quality parameters for one or more links between the base station and the relay, or (2) an availability status parameter indicating availability of the relay for the multi-path transmission.

Clause 46: A relay for a user equipment (UE) in a communication, the relay and the UE being attached to a base station, the relay comprising:
a memory storing an instruction; and
a processor configured to execute the instruction stored in the memory to:
receive, from the base station, a relay selection request;
receive, from the UE, one or more relay selection response messages, the one or more relay selection response messages including one or more link quality parameters for one or more links between the UE and the relay;
transmit, to the base station, the received one or more relay selection response messages; and
transmit, to the base station, one or more measurements on one or more signals transmitted between the base station and the relay, the one or more measurements comprising at least one of: (1) one or more link quality parameters for one or more links between the base station and the relay, or (2) one or more availability status parameters indicating availability of the relay for a multi-path transmission.

Clause 47: A non-transitory computer-readable medium storing instructions that are executable by one or more processors of a base station for communication, to perform a method for relay selection, the method comprising: receiving one or more discovery messages communicated between the UE and one or more candidate relays, the UE and the one or more candidate relays being attached to the base station;
receiving one or more measurements on one or more signals transmitted between the base station and the one or more candidate relays;
determining whether the one or more candidate relays satisfy relay selection criteria based on at least one of: the one or more discovery messages, or the one or more measurements; and
selecting, based on the determining, at least one relay from the one or more candidate relays for a multi-path transmission for the UE.

Clause 48: A non-transitory computer-readable medium storing instructions that are executable by one or more processors of a user equipment (UE) for communication, to perform a method, the method comprising:
receiving, from one or more candidate relays attached to the base station, one or more discovery messages, the one or more discovery messages comprising at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission;
transmitting, to the base station, the one or more discovery messages;
receiving, from the base station, information regarding at least one relay selected from the one or more candidate relays; and
triggering a multi-path transmission, the multi-path transmission comprising a direct path between the UE and the base station, and an indirect path between the UE and the base station via the selected at least one relay.

Clause 49: A non-transitory computer-readable medium storing instructions that are executable by one or more processors of a user equipment (UE) for communication, to perform a method, the method comprising:
transmitting, to the base station, a relay selection request such that the relay selection request is forwarded to one or more candidate relays attached to the base station;
transmitting, to the one or more candidate relays, one or more relay selection response messages, the one or more relay selection response messages comprising one or more link quality parameters for one or more links between the UE and the one or more candidate relays;
receiving, from the base station, information regarding at least one relay selected from the one or more candidate relays; and
triggering a multi-path transmission, the multi-path transmission comprising a direct path between the UE and the base station, and an indirect path between the UE and the base station via the selected at least one relay.

Clause 50: A non-transitory computer-readable medium storing instructions that are executable by one or more processors of a relay for a user equipment (UE) in a communication, to perform a method, the method comprising:
transmitting, to the UE, one or more discovery messages, the one or more discovery messages comprising at least one of: (1) one or more link quality parameters for one or more links between the UE and the relay, (2) an attachment status parameter indicating attachment of the relay to the base station, or (3) an availability status parameter indicating availability of the relay for multi-path transmission; and
transmitting, to the base station, one or more measurements on one or more signals transmitted between the base station and the relay, the one or more measurements comprising at least one of: (1) one or more link quality parameters for one or more links between the base station and the relay, or (2) an availability status parameter indicating availability of the relay for the multi-path transmission.

Clause 51: A non-transitory computer-readable medium storing instructions that are executable by one or more processors of a relay for a user equipment (UE) in a communication, to perform a method, the method comprising:
receiving, from the base station, a relay selection request;
receiving, from the UE, one or more relay selection response messages, the one or more relay selection response messages including one or more link quality parameters for a link between the UE and the relay;
transmitting, to the base station, the received one or more relay selection response messages; and
transmitting, to the base station, one or more measurements on one or more signals transmitted between the base station and the relay, the one or more measurements comprising at least one of: (1) one or more link quality parameters for one or more links between the base station and the relay, or (2) one or more availability status parameters indicating availability of the relay for a multi-path transmission.

1. A method for selecting at least one relay for a user equipment (UE) in a communication system, the method comprising:
receiving, by a base station, one or more discovery messages communicated between the UE and one or more candidate relays, the UE and the one or more candidate relays being attached to the base station;
receiving, by the base station, one or more measurements on one or more signals transmitted between the base station and the one or more candidate relays;
determining, by the base station, whether the one or more candidate relays satisfy relay selection criteria based on at least one of: the one or more discovery messages, or the one or more measurements; and
selecting, by the base station and based on the determining, at least one relay from the one or more candidate relays for a multi-path transmission for the UE.

2. The method of claim 1, wherein the one or more candidate relays comprise at least one of: one or more UEs that are different from the UE, or one or more road side units attached to the base station.

3. The method of claim 1, wherein the one or more discovery messages comprise at least one of: (1) one or more relay selection requests, or (2) one or more relay selection response messages indicating one or more responses to the one or more relay selection requests.

4. The method of claim 1, wherein the one or more discovery messages comprise at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission.

5. The method of claim 4, wherein the one or more link quality parameters for the one or more links between the UE and the one or more candidate relays comprise at least one of: one or more sidelink-reference signal received power (SL-RSRP) values measured on one or more sidelink signals transmitted between the UE and the one or more candidate relays, or one or more sidelink discovery-reference signal received power (SD-RSRP) values measured on the one or more discovery messages.

6. The method of claim 5, wherein the relay selection criteria comprise at least one of: (1) the one or more SL-RSRP values measured on the one or more sidelink signals transmitted between the UE and the one or more candidate relays exceed an SL-RSRP threshold, or (2) the one or more SD-RSRP values measured on the one or more discovery messages exceed a SD-RSRP threshold.

7. The method of claim 1, wherein the one or more measurements comprises at least one of: (1) one or more link quality parameters for one or more links between the base station the one or more candidate relays, or (2) one or more availability status parameters indicating availability of the one or more candidate relays for the multi-path transmission.

8. The method of claim 7, wherein the one or more link quality parameters for the one or more links between the base station and the one or more candidate relays comprise at least one of: one or more RSRP values measured on one or more signals transmitted between the base station and the one or more candidate relays, or one or more reference signal received quality (RSRQ) values measured on the one or more signals transmitted between the base station and the one or more candidate relays.

9. The method of claim 8, wherein the relay selection criteria comprise at least one of: (1) the RSRP values measured on the one or more signals transmitted between the base station and the one or more candidate relays exceed an RSRP threshold, or (2) the RSRQ values measured on the one or more signals transmitted between the base station and the one or more candidate relays exceed an RSRQ threshold.

10. The method of claim 1, wherein receiving the one or more discovery messages communicated between the UE and the one or more candidate relays further comprises:
receiving, from the UE, the one or more discovery messages that are obtained from the one or more candidate relays, the one or more discovery messages being one or more selection response messages and comprising at least one of: (1) one or more link quality parameters for one or more links between the UE and the one or more candidate relays, or (2) one or more attachment status parameters indicating attachment of the one or more candidate relays to the base station.

11. The method of claim 10, wherein receiving the one or more measurements on the one or more signals transmitted between the base station and the one or more candidate relays further comprises:
receiving, from the one or more candidate relays, the one or more measurements, the one or more measurements comprising at least one of: (1) one or more link quality parameters for one or more links between the base station the one or more candidate relays, or (2) one or more availability status parameters indicating availability of the one or more candidate relays for the multi-path transmission.

12. The method of claim 1, wherein receiving the one or more discovery messages communicated between the UE and the one or more candidate relays further comprises:
receiving, from the one or more candidate relays, the one or more discovery messages that are obtained from the UE, the one or more discovery messages being one or more selection response messages and comprising one or more link quality parameters for one or more links between the UE and the one or more candidate relays.

13. The method of claim 12, wherein receiving the one or more discovery messages communicated between the UE and the one or more candidate relays further comprises:
forwarding, to the one or more candidate relays, one or more relay selection requests received from the UE; and
receiving, from the one or more candidate relays, one or more relay selection response messages obtained from the UE in response to the one or more relay selection requests.

14. The method of claim 12, wherein receiving the one or more measurements on the one or more signals transmitted between the base station and the one or more candidate relays further comprises:
receiving, from the one or more candidate relays, the one or more measurements, the one or more measurements comprising at least one of: one or more link quality parameters for one or more links between the base station the one or more candidate relays, or (2) one or more availability status parameters indicating availability of the one or more candidate relays for the multi-path transmission.

15. The method of claim 1, further comprising:
transmitting, to the UE, the selected at least one relay to trigger the multi-path transmission at the UE, the multi-path transmission comprising a direct path between the UE and the base station, and an indirect path between the UE and the base station via the selected at least one relay.

16-23. (canceled)

24. A base station in a communication system, the base station comprising:
a memory storing an instruction; and
a processor configured to execute the instruction stored in the memory to:
receive one or more discovery messages communicated between the UE and the one or more candidate relays, the UE and the one or more candidate relays being attached to the base station;
receive one or more measurements on one or more signals transmitted between the base station and the one or more candidate relays;
determine whether the one or more candidate relays satisfy relay selection criteria based on at least one of: the one or more discovery messages, or the one or more measurements; and
select at least one relay from the one or more candidate relays for a multi-path transmission for the UE.

25. The base station of claim 24, wherein the one or more candidate relays comprise at least one of: one or more UEs that are different from the UE, or one or more road side units attached to the base station.

26. The base station of claim 24, wherein the one or more discovery messages comprise at least one of: (1) one or more relay selection requests, or (2) one or more relay selection response messages indicating one or more responses to the one or more relay selection requests.

27. The base station of claim 24, wherein the one or more discovery messages comprise at least one of: (1) one or more link quality parameters for one or more links between the UE and the one or more candidate relays, (2) one or more attachment status parameters indicating attachment of the one or more candidate relays to the base station, or (3) one or more availability status parameters indicating availability of the one or more candidate relays for the multi-path transmission.

28. The base station of claim 27, wherein the one or more link quality parameters for the one or more links between the UE and the one or more candidate relays comprise at least one of: one or more sidelink-reference signal received power (SL-RSRP) values measured on one or more signals transmitted between the UE and the one or more candidate relays, or one or more sidelink discovery-reference signal received power (SD-RSRP) values measured on the one or more discovery messages.

29-51. (canceled)
